(19) United States
(12) Patent Application Publication
(10) Pub. No.: US 2020/0093768 A1
(43) Pub. Date: Mar. 26, 2020

(54) METHOD FOR TREATING DIABETES

(71) Applicant: Huanggang Normal University, Huanggang (CN)

(72) Inventors: Shiming Li, Glastonbury, CT (US); Alexander Michael Gosslau, Highlands, NJ (US); Emmanuel Selvanayagam Zachariah, Highland Park, NJ (US)

(73) Assignee: Huanggang Normal University, Huanggang (CN)

(21) Appl. No.: 16/139,072

(22) Filed: Sep. 23, 2018

(51) Int. Cl. A61K 31/192; A61P 3/10; A61P 29/00

(52) U.S. Cl. CPC A61K 31/192; A61P 29/00; A61P 3/10

(57) ABSTRACT

Disclosed is a method of treating diabetes. The method includes administering an effective amount of a pharmaceutical composition to a subject in need thereof and the pharmaceutical composition contains ibuprofen. The ibuprofen can be administered at a dosage of 1-300 mg/kg per day and the pharmaceutical composition is administered for at least 30 days.

### FIELD OF THE INVENTION

The present invention relates generally to the field of disease therapeutics. Particularly, it relates to use of a therapeutic agent to treat diabetes in human or animal subjects. More specifically, the present invention provides ibuprofen as a therapeutic agent.

### BACKGROUND OF THE INVENTION

Type 2 diabetes (T2D) is one of the most escalating global health concerns (Bluher, Clin Sci (Lond). 130:1603-1614. 2016). Currently, at 425 million worldwide, T2D is projected to be at 629 million people by 2045 with a high proportion of the diabetic population still remaining undiagnosed according to the International Diabetes Federation (www.idforg). It is generally accepted that chronic inflammation is a key pathologic link between obesity and T2D. Dysfunctional lipid metabolism in hypertrophic adipose tissue gives rise to increased circulating free fatty acids (FFAs) leading to hyperlipidemia and lipid peroxidation. These dyslipidemic events cause an accumulation of necrotic, apoptotic and autophagic adipocytes (Boutens and Stienstra, Diabetologia. 59:879-894, 2016), followed by an infiltration of pro-inflammatory immune cells (Boutens and Stienstra, Diabetologia. 59:879-894, 2016). The accumulation of free radicals released by immunocompetent cells, or derived from conditions of hyperglycemia and dyslipidemia, are responsible for progression of T2D. In a vicious cycle, more reactive radicals formed by high glucose expedite an impairment of the insulin receptor, causing a further disconnection of the insulin cascade, thus leading to chronic hyperglycemia and insulin resistance (Boutens and Stienstra, Diabetologia. 59:879-894, 2016). Due to the connection between obesity and chronic inflammation, a dramatic rise in T2D is expected with an aging population and epidemic obesity (Bluher, Clin Sci (Lond). 130:1603-1614. 2016).

Two different types of macrophages contribute to inflammation in adipose tissue. M1 alternative type macrophages have mainly anti-inflammatory functions through IL-4, IL-10 and IL-13, and predominate in lean adipose tissue (Boutens and Stienstra, Diabetologia. 59:879-894, 2016). M2 classical macrophages induce inflammation through secretion of pro-inflammatory cytokines and chemokines. Hypertrophic adipose tissue derived from obese individuals is mainly infiltrated with M2 macrophages, visible as crown-like structures (Lee, Arch Pharm Res. 36:208-222, 2013). The release of TNF-α, IL-1β, IL-6 and ICAM-1 stimulate the inflammatory cascade by positive feedback mechanisms via NFκB and AP-1 signaling, to generate free radicals such as reactive nitrogen (RNS) and reactive oxygen species (ROS) (Bluher, Clin Sci (Lond). 130:1603-1614. 2016). Increased serine phosphorylation of insulin receptor substrates (e.g., IRS-1 and IRS-2) by JNK and NFκB signaling, in turn, inhibit the tyrosine kinase activity of the insulin receptor (Schenk et al., J Clin Invest. 118:2992-3002, 2008). An impairment of PI-3K/AKT insulin signaling causes a decrease of translocation and insertion of GLUT-4 leading to chronic hyperglycemia (Li et al., Sci Rep. 7:41289, 2017). Ultimately, the formation of advanced glycated endproducts (AGE) by reactive carbonyl species (RCS; e.g., methylglyoxal) and glycated proteins (e.g., HbA1c) will lead to cell, tissue and organ damage, subsequently causing nephropathy, cardiovascular disease, retinopathy, neuropathy or different cancers (Chawla et al., Nat Rev Immunol. 11:738-749, 2011).

Therapies for diabetes mostly involve control of hyperglycemia or insulin resistance. Metformin (1,1-dimethylbiguanide) is usually well-tolerated and considered to be the first-line antihyperglycemic drug treatment for T2D by mechanisms of increased cellular insulin sensitivity and suppression of hepatic glucose production (Romero et al., Am J Obstet Gynecol. 217:282-302, 2017). However, little attention has been paid to inflammatory pathways in T2D.

More recent research has shown that NSAIDs, such as acetylsalicylate (aspirin), salicylate and salsalate have been shown to reverse hyperglycemia, hyperinsulinemia, and dyslipidemia in obese mice as well as in patients with T2D. Ibuprofen (2-(4-Isobutylphenyl)propanoic acid) is widely used for treatment of pain, inflammation and fever (Davies, Clin Pharmacokinet. 34:101-154, 1998). Due to its safe and tolerability profile, it is the only NSAID approved for use in children over 3 months old (de Martino et al., Drugs. 77:1295-1311, 2017). Ibuprofen is a nonselective inhibitor of COX-1 and COX-2 (Davies, Clin Pharmacokinet. 34:101-154, 1998) and inhibits NFκB signaling to decrease the expression of inflammatory genes. Additionally, ibuprofen may activate PPAR-α and PPAR-γ and/or inhibit ribosomal S6 kinase 2 (Tegeder et al., FASEB J. 15:2057-2072, 2001).

Clearly, to find more therapies for diabetes patients is imperative as the current therapies available to patients are far from ideal. Ibuprofen is a proven drug. Yet to date, there are few studies of ibuprofen and diabetes. In 1983, a clinical pilot study using a low dosage of ibuprofen in hyperglycemic adults did not reveal major differences in glucose and insulin (Mork and Robertson, West J Med. 139:46-49, 1983).

### SUMMARY OF THE INVENTION

The present invention provides a method of using ibuprofen as a therapeutic agent to treat diabetes and its related syndromes. Through studies including application of a diabetic model, the NSAID agent exhibits an unexpectedly strong anti-inflammatory, anti-hyperglycemic, and anti-hyperlipidemic activities, thus providing a safe and effective way to treat the otherwise difficult metabolic disease. The study results suggest a potential therapeutic application of ibuprofen affecting different pathways leading to T2D.

One aspect of this invention relates to a method of treating diabetes. The method includes administering an effective amount of a pharmaceutical composition to a subject in need thereof and the pharmaceutical composition contains ibuprofen.

In the above method, the ibuprofen is administered at a dosage of 1-300 mg/kg per day. Other embodiment of the ibuprofen dosage administered per day can be 3-300 mg/kg, 5-300 mg/kg, 10-300 mg/kg, 10-200 mg/kg, 30-300 mg/kg, 30-200 mg/kg, 50-300 mg/kg, 50-200 mg/kg, 75-300 mg/kg, 75-200 mg/kg, 100-300 mg/kg, or 100-200 mg/kg. Preferably, the ibuprofen is administered at a dosage of 100 mg/kg per day.

Further, in the above method, the pharmaceutical composition is administered for at least 30 days.

One example of the diabetes is type 2 diabetes. On the other hand, the subject can be a human.

Another aspect of this invention relates to a method of increasing insulin sensitivity and glucose uptake. The method includes administering an effective amount of a pharmaceutical composition to a subject in need thereof and the pharmaceutical composition contains ibuprofen.

The ibuprofen can be administered at a dosage of 1-300 mg/kg per day.

Yet another aspect of this invention relates to a method for treating elevated HbA1c levels in a subject in need thereof. The method includes administering an effective amount of a pharmaceutical composition to said subject and the pharmaceutical composition contains ibuprofen.

Still another aspect of this invention relates to a method for treating insulin resistance syndrome. The method includes administering an effective amount of a pharmaceutical composition to a subject in need thereof and the pharmaceutical composition contains ibuprofen.

For both of the immediately above methods, the ibuprofen is preferably administered at a dosage of 1-300 mg/kg per day. Moreover, in the three additional methods introduced below, ibuprofen described therein is preferably administered at a dosage of 1-300 mg/kg per day. Additionally, other embodiment of the ibuprofen dosage administered per day can be 3-300 mg/kg, 5-300 mg/kg, 10-300 mg/kg, 10-200 mg/kg, 30-300 mg/kg, 30-200 mg/kg, 50-300 mg/kg, 50-200 mg/kg, 75-300 mg/kg, 75-200 mg/kg, 100-300 mg/kg, or 100-200 mg/kg.

Another aspect of this invention relates to a method for treating hyperglycemia. The method includes administering an effective amount of a pharmaceutical composition to a subject in need thereof. The pharmaceutical composition contains ibuprofen and is, advantageously, administered for at least 30 days.

One more aspect of this invention relates to a method for treating hyperlipidemia. The method includes administering an effective amount of a pharmaceutical composition to a subject in need thereof. Also, the pharmaceutical composition contains ibuprofen and is preferably administered for at least 30 days.

One additional aspect of this invention relates to a method for reducing chronic inflammation in a diabetic subject. The method includes administering an effective amount of a pharmaceutical composition to said diabetic subject, in which the pharmaceutical composition contains ibuprofen.

All the above-described methods of this invention can be applied to a mammal, e.g., a human or a rat.

The details of the invention are set forth in the drawing and the description below. Other features, objects, and advantages of the invention will be apparent to those persons skilled in the art upon reading the drawing and the description, as well as from the appended claims.

### BRIEF DESCRIPTION OF THE DRAWINGS

These drawings illustrate certain aspects of some of the embodiments of the present invention, and should not be used to limit or define the invention.

FIG. 1 is a diagram illustrating effects of ibuprofen and metformin on fasted and fed glucose levels.

FIG. 2 is a diagram that show effects of ibuprofen and metformin on glucose levels by the oral glucose tolerance test (OGGT).

FIG. 3 is a diagram or bar plot showing effects of ibuprofen and metformin on levels of glycated hemoglobin and insulin.

FIG. 4 is a diagram or bar plot showing effects of ibuprofen and metformin on levels of lipid related mediators.

FIG. 5 is a diagram or bar plot showing Effects of ibuprofen and metformin on levels of anti-inflammatory cytokines.

FIG. 6 is a diagram or bar plot showing effects of ibuprofen and metformin on the expression of inflammatory genes.

### DETAILED DESCRIPTION

Methods are provided for treating a subject having diabetes or other types of related symptoms. Aspects of the methods include administering to the subject an effective amount of a pharmaceutical composition that contains ibuprofen.

Before the present methods are described, it is to be understood that this invention is not limited to particular method described, as such may, of course, vary. It is also to be understood that the terminology used herein is for the purpose of describing particular embodiments only, and is not intended to be limiting, since the scope of the present invention will be limited only by the appended claims.

The subject methods are useful primarily for therapeutic purposes. Thus, as used herein, the term “treating” is used to refer to both prevention of disease, and treatment of a pre-existing condition. The treatment of ongoing disease, to stabilize or improve the clinical symptoms of the patient, is a particularly important benefit provided by the present invention. Such treatment is desirably performed prior to loss of function in the affected tissues including the cardiovascular system and its surrounding tissues. For example, treatment of a cancer patient may be reduction of tumor size, elimination of malignant cells, or the prevention of relapse in a patient who has been put into remission.

The terms “inhibiting,” “reducing,” or “prevention,” or any variation of these terms, when used in the claims and/or the specification includes any measurable decrease or complete inhibition to achieve a desired result.

On the other hand, the terms “determining,” “measuring,” and “assessing,” and “assaying” are used interchangeably and include both quantitative and qualitative determinations.

The terms “subject,” “host,” “patient,” and “individual” are used interchangeably herein to refer to any mammalian subject for whom diagnosis or therapy is desired, particularly humans. Other subjects may include cattle, dogs, cats, guinea pigs, rabbits, rats, mice, horses, and so on.

The terms “cell,” and “cells,” and “cell population,” used interchangeably, intend one or more mammalian cells. The term includes progeny of a cell or cell population. Those skilled in the art will recognize that “cells” include progeny of a single cell, and there are variations between the progeny and its original parent cell due to natural, accidental, or deliberate mutation and/or change.

An “effective amount” is an amount sufficient to effect beneficial or desired clinical results. An effective amount can be administered in one or more administrations. For purposes of this invention, an effective amount of reagent antibodies is an amount that is sufficient to diagnose, palliate, ameliorate, stabilize, reverse, slow or delay the progression of the disease state.

ZDF rat model animals have significantly higher body weight, BMI and food intake with higher plasma levels of glucose in the absorptive and post absorptive states. In addition, ZDF animals have significantly higher HbA1c levels, triglycerides, free fatty acids, cholesterol, HDL, and slightly increased levels of LDL, as compared to lean control rats. Moreover, the inflammatory cytokine TNF-α was upregulated in epididymal adipose tissue and plasma, confirming a central role in the adipocyte inflammatory cascade (Chawla et al., Nat Rev Immunol. 11:738-749, 2011). Thus, ZDF rats authenticate obesity as a leading cause for T2D and damaging effects on various organs. There have been controversial reports on the role of IL-6 signaling in obesity-related insulin resistance (Chen et al., Int J Endocrinol. 2015:1-9, 2015).

Systemic inflammation is well accepted as causal in T2D development. It is well documented that a more favorable glycemic, lipidemic as well as inflammatory profile in a normal weight population have a positive impact on human health. These relationships are evident in studies showing that caloric restriction can induce an extension of life span in mammals (Sohal and Weindruch, Science. 273:59-63, 1996).

Metformin is a widely used T2D drug which significantly decreased fasted blood glucose levels and improved glucose tolerance in ZDF rats. Previous studies suggest anti-inflammatory effects by metformin as indicated by a decline in IL-1β, COX-2, and iNOS as well as increase in IL-10, respectively (Kelly et al., J Biol Chem. 290:20348-20359, 2015). However, possible anti-inflammatory effects of metformin have not been confirmed in clinical studies (Caballero et al., J Clin Endocrinol Metab. 89:3943-3948, 2004).

Results obtained from the study showed novel effects of ibuprofen against hyperglycemia and dyslipidemia as well against inflammation in the obese rat ZDF model. In addition, weight reduction in ZDF rats treated with ibuprofen may add to the antidiabetic effects. These results will have to be consolidated in more predictable long-term and clinical studies required to clarify the role of ibuprofen against pathological T2D conditions. For an aging population with epidemic obesity rates, ibuprofen may be useful as an adjunct therapy in conjunction with life style changes for better weight management to avoid T2D symptoms.

### Materials and Methods

### Materials and Chemicals

For RNA isolation from whole blood and epididymal adipose tissue, Trizol reagent and RNeasy™ Total RNA Kit or RNeasy™ Lipid Mini Kit (Qiagen, Chatsworth, Calif.) was used. Oligo-dT, dNTPs, Superscript™ II reverse transcriptase were purchased from Invitrogen, Life Technologies (Grand Island, N.Y.). TaqMan qPCR probes, primers and master mix were from Applied Biosystems, Life Technologies (Grand Island, N.Y.). Other chemicals were purchased from Sigma (St. Louis, Mo.).

### Animals and Treatment

Zucker diabetic fatty (ZDF) rats, based on a missense mutation in the leptin receptor gene (Phillips et al., 1996) and control animals were studied at Charles River Laboratories (CRL). 24 six week old male ZDF were used. At 10 weeks of age, fed glucose (via glucometer) was assessed and 24 animals with blood glucose levels ≥250 mg/dL were selected along with 8 age matched lean controls. Rats were singly housed on a normal light cycle in the animal facility and received a control diet 5008 (LabDiets, changed weekly) for the duration the study. All protocols were approved by the Institutional Animal Care and Use Committee (IACUC; Piedmont research center).

For experiments, the 24 ZDF rats were randomized and combined with the lean control group into four different cohorts by HbA1c levels: group 1 (Lean ZDF control treated with vehicle (0.5% HPMC+0.2% Tween)); group 2 (ZDF control treated with vehicle); group 3 (ZDF treated with metformin (250 mg/kg)); group 4 (ZDF treated with ibuprofen (100 mg/kg)). Oral gavage doses were formulated weekly, and released from the pharmacy in daily aliquots for dosing. Animals were gavaged once daily for 30 days at 10 mL/kg. Rats were weighted twice a week and food intake was recorded weekly. Body mass index (BMI) was determined after animals were weighed using animal length (tip of the nose to the tip of the tail). Fed (at 8 hrs, prior to test article dosing) and 5 hrs fasted (at 13 hrs) blood glucose was checked at Baseline, Day 8, 15, and 22, and 28. On study day 29, an oral glucose tolerance test was conducted on overnight fasted animals. Food was returned to all animals following the final time point.

On study day 30, blood was collected at CRL. HbA1c was measured at 8 hrs on a drop of tail whole blood and then animals were fasted. At 11 hrs, animals were dosed per normal and euthanized at 13 hrs by CO₂ asphyxiation. Blood was withdrawn by cardiac puncture and 1 mL of whole blood placed into cryo vials for RNA extraction. Remaining blood was centrifuged (at 2200×g for 10 minutes at 22° C.) and serum (500 uL) pipetted into 96 well plates on dry ice for analysis of the following: insulin, adiponectin, clinical chemistry panel with lipid parameters, and the inflammatory panel. Liver, epididymal fat, kidney, heart and spleen were collected and a representative piece was snap frozen. 100 mg of tissue was transferred into 1.5 mL cold RNA later tubes and stored overnight at 4° C. before being moved to −20° C.

### HbA1c Analysis

Glycated hemoglobin (HbA1c) levels in venous whole blood were determined at CRL using the HbA1c Now test kit (Bayer, Whippany, N.J.). HbA1c was measured in treatment groups by immunoassay with anti-HbA1c using tail nick whole blood.

### Oral Glucose Tolerance Test

On study day 29 an oral glucose tolerance test (OGTT) was conducted on overnight fasted animals at CRL. All animals had been dosed per normal daily routine at 8 hrs. One hour later animals were gavaged with glucose at 2 g/kg. Whole blood glucose sampling occurred at the following times (min) relative to glucose dose: 0, 15, 30, 60, 90 and 120 min and were determined using a veterinary glucometer (Alpha Trak, Abbott Laboratories, Abbott Park, Ill.).

### Clinical Chemistry Analysis

Plasma analysis of insulin, adiponectin, cytokines and a full clinical chemistry panel with lipid parameters was performed at CRL using commercially available ELISA and colorimetric kits.

TaqMan qPCR Analysis

For gene expression analysis by TaqMan qPCR, five inflammatory surrogate genes (COX-2, TNF-α, ICAM-1, IL-1β, and IL-6) were previously selected and validated in cell-based, animal and clinical studies by whole genome Affymetrix and custom-made Oligo microarrays (Gosslau et al., 2011). RNA was isolated from whole blood samples with Trizol reagent, followed by chloroform and isopropanol extraction. Total RNA was precipitated using the RNeasy™ (Qiagen, Chatsworth, Calif.) for whole blood or RNeasy™ Lipid Mini Kit for epididymal adipose tissue. Total RNA was reverse transcribed using standard protocols and reagents from Invitrogen, Life Technologies (Grand Island, N.Y.). TaqMan qPCR was run on a Roche 480 Lightcycler (Roche Life Science, Indianapolis, Ind.) for 50 cycles with concentrations ranging from 0.01 to 100 ng for the standard curve. Gene expression of COX-2 (Rn01483828_m1), TNF-α (Rn01525859_g1), ICAM-1 (Rn00564227_m1), IL-1β (Rn00580432_m1), IL-6 (Rn01410330_m1), and GAPDH (Rn01775763_g1) were analyzed using probes, primers and master mix from Applied Biosystems (Life Technologies). After normalization to GAPDH, gene expression was expressed either as delta CT mean values+/−standard deviation or as delta-delta ct values to show the comparison with ZDF vehicle controls.

### Statistics

Results are expressed as mean values+standard deviation for the different treatment groups (n=8 for each group). Statistical comparisons of data were performed using the student's t-test.

### EXAMPLES

### Example 1: Effects of Ibuprofen and Metformin on Body Weight and Food Intake

Food intake, body weight and BMI were significantly lower in lean control rats compared to the ZDF control group (Table 1). All treatment groups showed a decline in body weight between day 25 and day 29 due to the overnight fasting on day 28. Ibuprofen induced a decline in body weight between day 8 and 26 which correlated with lower food intake. This was significant when compared with the ZDF control rats but to a lesser extent when compared to the lean group. However, the decline in body weight and food intake in response to ibuprofen treatment reached significance on day 8 (p<0.05). Chronic treatment with metformin did not cause any significant changes in body weight, food intake or BMI, respectively. Furthermore, the chemical analysis of blood and rat tissues confirmed that ibuprofen and metformin treatments were non-toxic and well tolerated (data not shown).

TABLE 1

| | A) Body Weight_Day | | | | | | | | |
|---|---|---|---|---|---|---|---|---|---|
| | 1 | 4 | 8 | 11 | 15 | 19 | 22 | 26 | 29 |
| LEAN_MEAN | 288.8 | 295.3 | 307.5 | 313.3 | 315.1 | 322.8 | 331.4 | 337.5 | 325.5 |
| LEAN_SD | 15.0 | 14.6 | 16.8 | 17.0 | 16.2 | 16.1 | 18.2 | 19.0 | 16.5 |
| ZDF_MEAN | 388.6 | 384.0 | 400.1 | 400.5 | 392.3 | 399.4 | 402.9 | 408.4 | 377.5 |
| ZDF_SD | 19.5 | 25.5 | 22.7 | 23.3 | 27.2 | 23.3 | 26.1 | 25.1 | 28.4 |
| ZDF + MET_MEAN | 376.6 | 377.8 | 388.5 | 396.1 | 390.9 | 400.8 | 399.9 | 405.6 | 378.9 |
| ZDF + MET_SD | 20.1 | 23.7 | 21.4 | 24.8 | 27.5 | 26.7 | 29.1 | 28.8 | 28.6 |
| ZDF + IBU_MEAN | 386.5 | 370.8 | 376.4** | 379.4* | 377.9 | 376.3* | 385.8 | 380.6* | 359.0 |
| ZDF + IBU_SD | 15.8 | 15.3 | 16.6 | 17.0 | 20.3 | 28.7 | 23.4 | 27.3 | 26.6 |

| | B) Food Intake_Day | | | | | C) BMI_DAY | | | | |
|---|---|---|---|---|---|---|---|---|---|---|
| | 8 | 15 | 22 | 28 | | 1 | 8 | 15 | 22 | 29 |
| LEAN_MEAN | 169.1 | 170.6 | 145.9 | 151.5 | LEAN_MEAN | 1.95 | 2.05 | 1.98 | 2.06 | 1.95 |
| LEAN_SD | 11.7 | 46.6 | 6.3 | 16.4 | LEAN_SD | 0.08 | 0.11 | 0.10 | 0.12 | 0.10 |
| ZDF_MEAN | 277.4 | 292.4 | 305.6 | 289.4 | ZDF_MEAN | 2.61 | 2.63 | 2.46 | 2.52 | 2.37 |
| ZDF_SD | 18.5 | 19.7 | 21.9 | 10.4 | ZDF_SD | 0.07 | 0.08 | 0.12 | 0.14 | 0.13 |
| ZDF + MET_MEAN | 255.1 | 309.9 | 299.1 | 278.8 | ZDF + MET_MEAN | 2.60 | 2.57 | 2.47 | 2.52 | 2.39 |
| ZDF + MET_SD | 23.2 | 31.8 | 17.0 | 28.5 | ZDF + MET_SD | 0.07 | 0.15 | 0.08 | 0.09 | 0.08 |
| ZDF + IBU_MEAN | 225.4** | 258.8* | 233.5* | 239.3* | ZDF + IBU_MEAN | 2.67 | 2.51* | 2.44 | 2.47 | 2.28 |
| ZDF + IBU_SD | 40.2 | 33.7 | 49.7 | 45.5 | ZDF + IBU_SD | 0.10 | 0.09 | 0.13 | 0.14 | 0.18 |

### Example 2: Effects of Ibuprofen and Metformin on Glucose Homeostasis

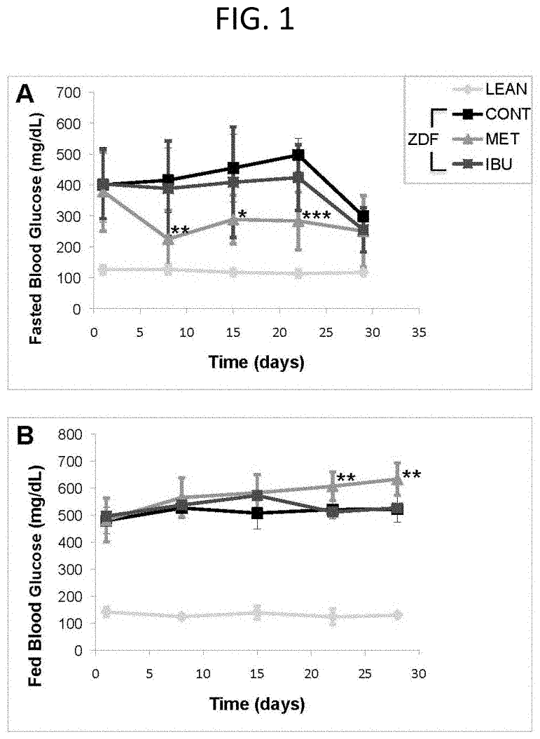

In the next set of experiments, effects of ibuprofen and metformin on glucose-related parameters analyzed. FIG. 1A shows a significant decrease in fasted blood glucose levels after 8 days of metformin treatment in ZDF rats. The overnight fasting on day 28, however, induced a decline of blood glucose levels to around 250 mg/dL on day 29 in all treatment groups. Lean control animals did not show major changes in glucose levels throughout the study. Ibuprofen treatment of rats did reduce fasting blood glucose levels but this was not significant. Fed glucose levels remained fairly constant throughout the different treatment groups with the exception of the metformin (250 mg/Kg) which showed significant higher levels on day 22 and 28 (FIG. 1B).

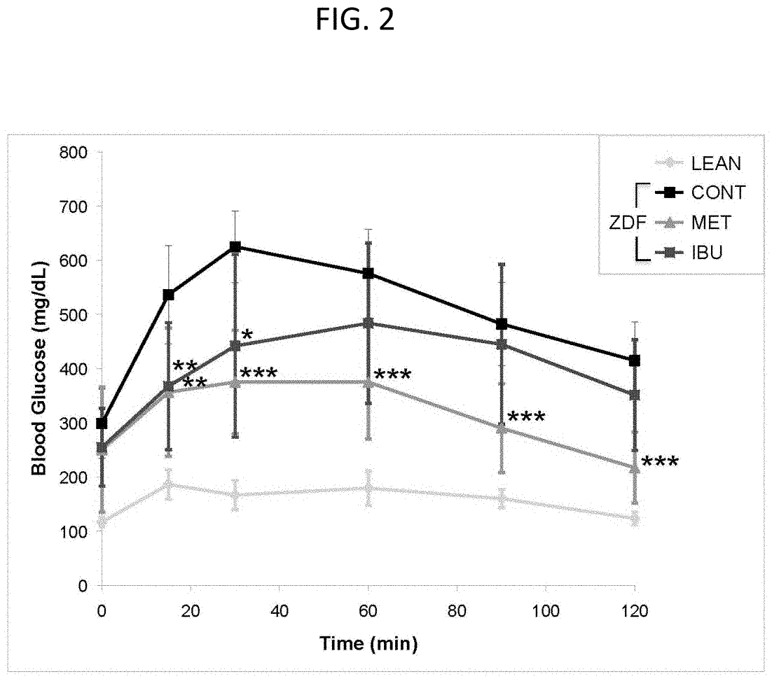

An oral glucose tolerance test (OGTT) was performed on day 29 of the study (FIG. 2). Metformin, as expected induced a significant decline in blood glucose in a time-dependent manner as compared to the ZDF vehicle control starting after 15 min (p<0.01). Ibuprofen treatment also caused a significant decrease in blood glucose 15 min (p<0.01) and 30 min (p<0.05) after the glucose challenge, but to a lesser degree as compared to metformin. In lean controls, it was observed a similar pattern of glucose clearance of significant smaller amounts of blood glucose.

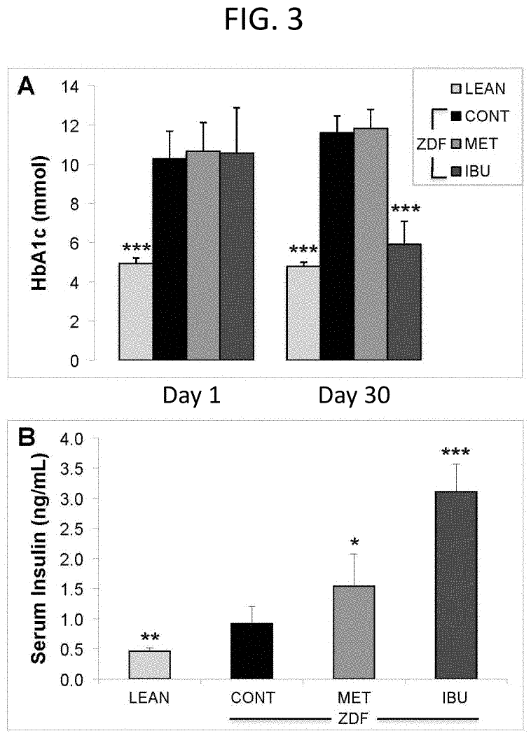

Glycated hemoglobin (HbA1c) was compared for all treatment groups at day 1 and day 30 (FIG. 3A). The lean control group showed significant lower levels of HbA1c on day 1 and day 30 as compared to ZDF vehicle control animals (p<0.001). Metformin did not significantly affect HbA1c levels after 30 days of treatment as compared to the ZDF controls. Significantly, ibuprofen attenuated the glycation of hemoglobin following 30 days after treatment (p<0.001). Analysis of plasma insulin levels on day 30 of the study revealed significant lower levels of insulin in lean controls as compared to ZDF rat treatment groups (FIG. 3B). Metformin treated ZDF rats showed significantly higher levels of insulin release (p<0.05) although the concentrations of insulin in the ibuprofen treated rats were higher (3.11 ng/mL) as compared to metformin treated animals (1.54 ng/mL) and significant when compared to ZDF controls (p<0.001).

### Example 3: Effects of Ibuprofen and Metformin on Fat Metabolism

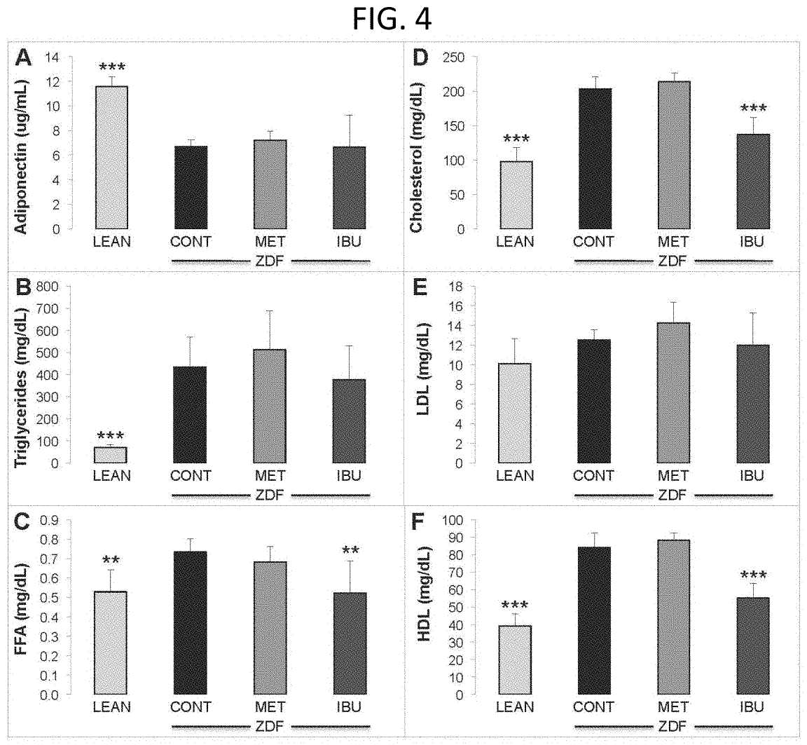

Significantly higher adiponectin levels were determined in plasma derived from the lean control group (11.6 ug/mL) as compared to ZDF controls (6.7 ug/mL), but not in the metformin or ibuprofen treated animals after 30 days (FIG. 4A). There was a significant reduction (p<0.001) in the levels of triglycerides (TGs) in the lean control animals (around 70 mg/dL) when compared to the ZDF control animals (around 434 mg/dL) but not in the metformin or ibuprofen treated animals (FIG. 4B). Free fatty acids (FFA) were reduced in plasma derived from lean control rats (p<0.01) as compared to the ZDF rats (FIG. 4C). Significantly, ibuprofen reduced FFAs to levels of lean control animals (p<0.01). A reduction of FFAs was also observed in metformin treated animals, but in a non-significant manner. Fasting cholesterol and HDL, but not LDL, were significantly reduced in the lean control group (FIGS. 4D-F) compared with ZDF control rats. No significant differences were observed for metformin treated rats. However, whereas no significant changes were observed for LDL, ibuprofen induced a strong decline in cholesterol and HDL (both with p<0.001) to levels only slightly higher as compared to lean controls.

### Example 4: Effects of Ibuprofen and Metformin on Inflammatory Mediators in Adipose Tissue and Whole Blood

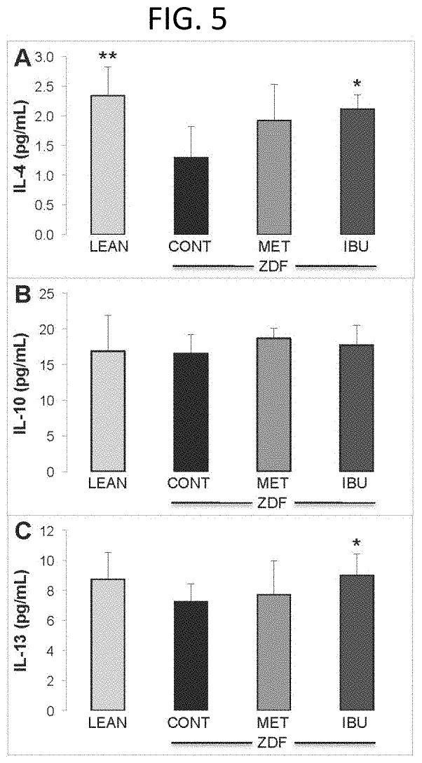

Previous studies have suggested a link between systemic inflammation. The expression of inflammatory genes in blood and epididymal adipose tissue was next analyzed. A panel of three anti-inflammatory cytokines in whole blood was quantified by ELISA analysis as a measure for systemic inflammation (FIG. 5). IL-4, IL-10 and IL-13 are involved in the reduction of the inflammatory response through inhibition of NFκB signaling and cytokine release from macrophages. IL-4 was significantly increased in lean controls (p<0.01) and ibuprofen treated ZDF rats (p<0.05) as compared to ZDF controls (FIG. 5A). IL-4 did increase also in metformin treated animals, but in a non-significant manner. No significant changes in IL-10 plasma levels were observed in lean controls, metformin and ibuprofen treated rats (FIG. 5B). IL-13 levels increased in response to ibuprofen treatment in ZDF rats (p<0.05), whereas the increase in the lean control group was non-significant (FIG. 5C).

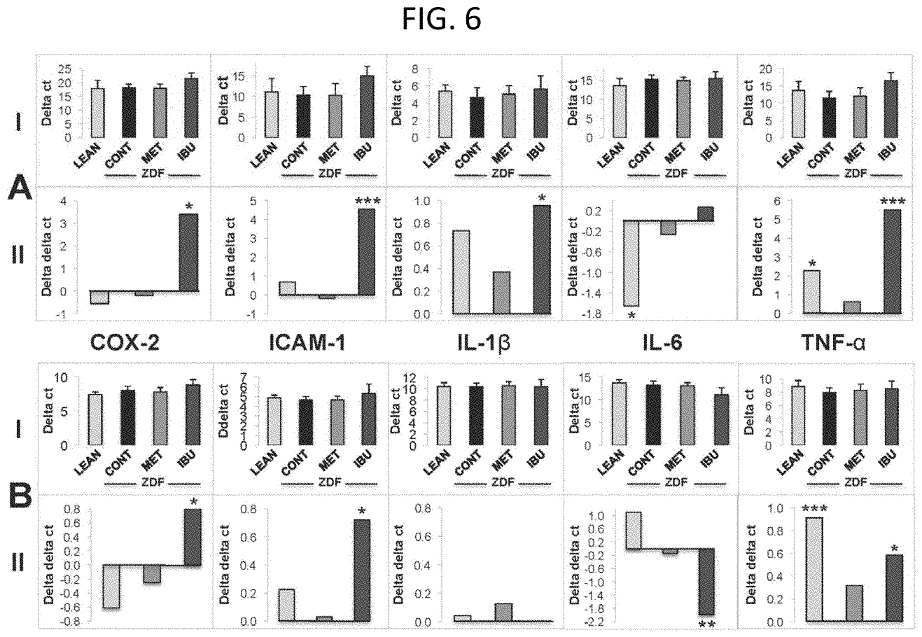

In the next set of experiments, the expression of a subset of inflammatory genes were quantified in whole blood (FIG. 6A) and epididymal fat tissue (FIG. 6B): cyclooxygenase-2 (COX-2), intracellular adhesion molecule-1 (ICAM-1), interleukin-1β (IL-1β), interleukin-6 (IL-6) and tumor necrosis factor-α (TNF-α) by TaqMan qPCR analysis. Gene expression levels were normalized to GAPDH (Delta ct values+/−SD indicated as “I”) or normalized to the ZDF controls (Delta delta ct values indicated as “II”) as described in the “Research Design and Methods” section. The expression of COX-2 in blood derived from lean control rats was similar to that of ZDF control animals (FIG. 6A, I). In contrast, ibuprofen treatment induced a significant down-regulation of COX-2 (p<0.05), as demonstrated by higher delta ct values. Similarly, there were no differences for ICAM-1 between lean and ZDF controls or metformin treated animals. However, ZDF rats treated with ibuprofen showed a drastic down-regulation of ICAM-1 (p<0.001). Furthermore, the inhibition of IL-1β by ibuprofen treatment was significant as compared to ZDF controls (p<0.05) in contrast to lean controls and ZDF rats treated with metformin. IL-6 was significantly up-regulated in lean control rat blood (p<0.05) but not in metformin or ibuprofen treated animals. Finally, a down-regulation of TNF-α was observed in lean control animals (p<0.05) but was more significantly reduced by ibuprofen (p<0.001).

The analysis of these genes in epididymal fat tissue (FIG. 6B) produced similar trends. COX-2 expression was significantly down-regulated in ibuprofen treated animals (p<0.05) but not in fat tissue from the lean control and metformin treated group. A similar pattern of gene regulation was observed in whole blood (A) and epididymal fat tissue (B) for ICAM-1, with ibuprofen significantly downregulating ICAM-1 (p<0.05), although to a lesser level as compared to whole blood. No differences throughout the treatment groups were observed for IL-1β expression in fat tissue. The regulation of IL-6 was different in fat tissue as compared to blood. Ibuprofen treatment significantly upregulated IL-6 (p<0.001) in contrast to the other groups. As observed for whole blood, TNF-α was down-regulated in lean control animals as compared to the ZDF controls but to a larger degree (p<0.001). Again, ibuprofen treatment significantly down-regulated TNF-α expression in epididymal fat (p<0.05), although this effect was greater in whole blood.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed. To the extent a definition of a term set out in a document incorporated herein by reference conflicts with the definition of a term explicitly defined herein, the definition set out herein controls.

1. A method of treating diabetes, comprising administering an effective amount of a pharmaceutical composition to a subject in need thereof, wherein:
the pharmaceutical composition comprises ibuprofen;
the ibuprofen is administered at a dosage of 1-300 mg/kg per day; and
the pharmaceutical composition is administered for at least 30 days.

2. (canceled)

3. The method of claim 1, wherein the ibuprofen is administered at a dosage of 100 mg/kg per day.

4. (canceled)

5. The method of claim 1, wherein the diabetes is type 2 diabetes.

6. The method of claim 1, wherein the subject is a human.

7. A method of increasing insulin sensitivity and glucose uptake, comprising administering an effective amount of a pharmaceutical composition to a subject in need thereof, wherein:
the pharmaceutical composition comprises ibuprofen;
the ibuprofen is administered at a dosage of 1-300 mg/kg per day; and
the pharmaceutical composition is administered for at least 30 days.

8. The method of claim 7, wherein the ibuprofen is administered at a dosage of 100 mg/kg per day.

9. The method of claim 7, wherein the method is further for treating elevated HbA1c levels in a subject in need thereof.

10. (canceled)

11. The method of claim 7, wherein the method is further for treating insulin resistance syndrome.

12. (canceled)

13. The method of claim 7, wherein the method is further for treating hyperglycemia.

14-15. (canceled)

16. The method of claim 7, wherein the method is further for treating hyperlipidemia.

17-18. (canceled)

19. A method for reducing chronic inflammation in a diabetic subject, comprising administering an effective amount of a pharmaceutical composition to said diabetic subject, wherein:
the pharmaceutical composition comprises ibuprofen;
the ibuprofen is administered at a dosage of 1-300 mg/kg per day; and
the pharmaceutical composition is administered for at least 30 days.

20. The method of claim 19, wherein the ibuprofen is administered at a dosage of 100 mg/kg per day.
